(19) United States
(12) Patent Application Publication
(10) Pub. No.: US 2010/0023030 A1
(43) Pub. Date: Jan. 28, 2010

(54) SURGICAL FASTENER DEVICES AND METHODS FOR THEIR MANUFACTURE AND USE

(76) Inventor: LEONARD REMIA, FT LAUDERDALE, FL (US)

Correspondence Address: RICHARD S. ROSS, ESQ., 4801 S. UNIVERSITY DR., # 237, FT. LAUDERDALE, FL, 33328, US

(21) Appl. No.: 12/505,511

(22) Filed: Jul. 19, 2009

Related U.S. Application Data
(60) Provisional application No. 61/083,390, filed on Jul. 24, 2008.

(51) Int. Cl. A61B 17/08

(52) U.S. Cl. 606/151

(57) ABSTRACT

The present invention is directed towards surgical fastener devices that combine a bioabsorbable component capable of being absorbed by recipient biological tissue such as bone over timer along with an integral biologically inert metallic threaded component that is intended to be permanently retained in recipient biological tissue such as bone. Surgical fasteners may also be provided in various embodiments according to the present invention in which bioabsorbable components of a desired length and/or width are fabricated may be attached at the time of deployment to biologically inert metallic components of a desired length and/or width.

### CROSS REFERENCE TO PROVISIONAL PATENT APPLICATION

Priority claimed to provisional application Ser. No. 61/083,390 filed Jul. 24, 2008.

### FIELD OF THE INVENTION

The present invention relates to the field of surgical fastener devices and method for their manufacture and use. Among the preferred embodiments of the present invention are improvements in the design and deployment of biometal screws particularly applicable to orthopedic surgery and the treatment of soft tissue and/or osseous defects.

### BACKGROUND OF THE INVENTION

Surgical fasteners are often placed in surgical procedures to attach a biological tissue or implant to adjacent biological tissues or implants. Such surgical fasteners may be used to attach a desired soft tissue structure such as a tendon to a bone or osseous implant structure to restore, approximate, or maintain physiologic function of a limb or joint. Surgical fasteners may also be placed to attach a plate or other implant to a bone. Furthermore, surgical fasteners may be placed to attach a bone fragment or graft to a defect in bone.

The design and structure of surgical fasteners such as screws, pins, nails, and rods has evolved from purely metallic devices to include bioabsorbable materials. The use of bioabsorbable material screws (“bioscrews”) is preferred because such screws eliminate the need of having to subsequently remove the hardware. The use of bioscrews also reduces the occurrence of tissue and suture laceration or compromise by the screw threads. Another advantage of the bioscrew is MRI compatability.

Existing surgical fastener systems are either composed of bioabsorbable materials, or are composed of conventional metallic designs. An exemplary bioabsorbable interference bone fixation screw is disclosed in U.S. Pat. No. 5,470,334. Additional exemplary bioabsorbable materials are disclosed in U.S. Pat. No. 4,968,317.

However, in certain surgical applications, it is desirable to have a permanently indwelling metallic fastener. Examples of such circumstances where a non-absorbable component is desirable include, but are not limited to, the attachment of tendons to bone or implant surfaces or the attachment of surgical plates to bone.

In such circumstances, it would be preferable to combine the advantages of a bioabsorbable surgical fastener with the advantages of a biologically inert metallic fastener such as stainless steel, titanium, other biologically inert metals, or alloys thereof.

### SUMMARY OF THE INVENTION

Surgical fastener devices according to the present invention are provided with one or more improvements that combine a bioabsorbable component with a biologically inert metallic component.

One aspect of the present invention is directed towards novel designs for biometal screws that provide a bioabsorbable component capable of being absorbed by recipient biological tissue such as bone over time, along with an integral biologically inert metallic threaded component that is intended to be permanently retained in recipient biological tissue such as bone.

In various embodiments according to the present invention, surgical fasteners may be provided in which bioabsorbable components of desired length and/or width are fabricated in an integral attachment to biologically inert metallic components of desired length and/or width.

In other embodiments according to the present invention, surgical fasteners may be provided in which bioabsorbable components of desired length and/or width are fabricated may be attached at the time of deployment to biologically inert metallic components of desired length and/or width.

The preceding descriptions are presented only as exemplary applications of the devices and methods according to the present invention.

### BRIEF DESCRIPTION OF THE DRAWINGS

FIG. 1 is a perspective view of an embodiment of a biometal surgical fixation screw according to the present invention.

FIG. 2 is a detailed view of an embodiment of a bioabsorbable component of a biometal surgical fixation screw according to the present invention.

FIG. 3 is a detailed view of an embodiment of a metallic threaded component of a biometal surgical fixation screw according to the present invention.

FIG. 4 is a perspective view of an embodiment of a calibrated depth gauge according to the present invention.

FIG. 5 is a perspective view of an embodiment of a biometal surgical fixation screw according to the present invention securing a tendon graft in a tibial tunnel.

### DETAILED DESCRIPTION OF DISCLOSED EMBODIMENTS

The present invention may be understood more readily by reference to the following detailed description of the preferred embodiments of the invention and the examples included herein. However, before the preferred embodiments of the devices and methods according to the present invention are disclosed and described, it is to be understood that this invention is not limited to the exemplary embodiments described within this disclosure, and the numerous modifications and variations therein that will be apparent to those skilled in the art remain within the scope of the invention disclosed herein. It is also to be understood that the terminology used herein is for the purpose of describing specific embodiments only and is not intended to be limiting.

Unless otherwise noted, the terms used herein are to be understood according to conventional usage by those of ordinary skill in the relevant art. In addition to the definitions of terms provided below, it is to be understood that as used in the specification and in the claims, “a” or “an” can mean one or more, depending upon the context in which it is used.

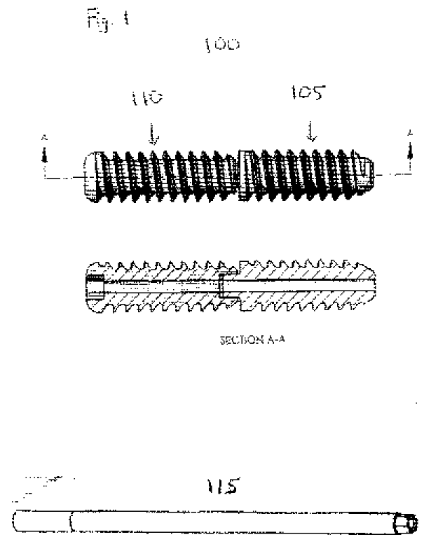

Referring now in more detail to the drawings, in which like numerals indicate like elements throughout the several views, FIG. 1 shows an embodiment of a biometal surgical fixation screw 100 according to the present invention, comprising a distal bioabsorbable threaded component 105 attached to a proximal metallic threaded component 110, with said biometal surgical fixation screw 100 being turned by a keyed detachable rotational deployment tool 115. Both screws have a continuous uniform inner diameter that spans the length of each screw as shown in Section A-A. In another embodiment, the continuous uniform inner diameter is keyed in a hexagonal shape; or may also be a continuous hexagonal taper, wider at the proximal end of the metallic screw and narrower at the distal end of the bioabsorbable screw. The keyed detachable rotational deployment tool may then be extended with a matching shape of the inner diameter (either uniform inner or tapered) of the biometal screw.

Biometal surgical fixation screws 100 according to the present invention may range in width from about 1 to about 30 mm; more preferably, certain embodiments of biometal surgical fixation screws 100 according to the present invention may range from about 3 to about 15 mm in width.

Distal bioabsorbable threaded components 105 of biometal surgical fixation screws 100 according to the present invention may vary in length from about 1 to about 100 mm in length; more preferably, certain embodiments of such distal bioabsorbable threaded components 105 may range from about 10 to about 30 mm in length.

Proximal metallic threaded components 110 of biometal surgical fixation screws 100 according to the present invention may vary in length from about 1 to about 100 mm in length; more preferably, certain embodiments of such proximal metallic threaded components 110 may range from about 10 to about 30 mm in length. Proximal metallic threaded components 110 of biometal surgical fixation screws 100 according to the present invention may comprise stainless steel, titanium, aluminum, other biologically inert metals, or alloys thereof.

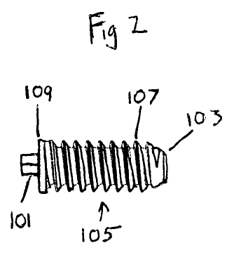

FIG. 2 provides a detailed view of the embodiment of a bioabsorbable threaded component 105 of FIG. 1, in which an absorbable body comprises a distal tip 103, a proximal end 109, and outer threads 107. In the embodiment shown in FIG. 2, the proximal end of the bioabsorbable threaded component 105 is an axial male keyed projection 101. A continuous uniform inner diameter that spans the entire length of the screw is shown in Section A-A of FIG. 1.

A bioabsorbable threaded component 105 according to the present invention may be fabricated from any bioabsorbable material of sufficient hardness to allow its use as a surgical fastener. Exemplary bioabsorbable materials may include, but are not limited to, bioabsorbable or biodegradable polymers or copolymers having an absorption or degradation time selected in accordance with the anticipated healing time for the fixated tissue. Table I set forth herein lists exemplary polymers (and copolymers and terpolymers thereof) suitable for bioabsorbable threaded component 105, and these polymers are all biodegradable into water-soluble, non-toxic materials that may be safely physiologically eliminated by the body of a recipient mammalian patient. Although the illustrative polymers are normally linear, suitable cross linked resins may also be prepared therefrom.

TABLE 1
Resorbable polymers

Polymer
Polyglycolide (PGA)
Copolymers of glycolide:
Glycolide/L-lactide copolymers (PGA/PLLA)
Glycolide/trimethylene carbonate copolymers (PGA/TMC)
Polylactides (PLA)
Stereocopolymers of PLA:
Poly-L-lactide (PLLA)
Poly-DL-lactide (PDLLA)
L-lactide/DL-lactide copolymers
Copolymers of PLA:
Lactide/tetramethylglycolide copolymers
Lactide/trimethylene carbonate copolymers
Lactide/σ-valerolactone copolymers
Lactide/ε-caprolactone copolymers
Polydepsipeptides
PLA/polyethylene oxide copolymers
Unsymmetrically 3,6-substituted poly-1,4-dioxane-2,5-
diones
Poly-β-hydroxybutyrate (PHBA)
PHBA/γ-hydroxyvalerate copolymers (PHBA/HVA)
Poly-β-hydroxypropionate (PHPA)
Poly-p-dioxanone (PDS)
Poly-σ-valerolactone
Poly-ε-caprolactone
Methylmethacrylate-N-vinyl pyrrolidone copolymers
Polyesteramides
Polyesters of oxalic acid
Polydihydropyrans
Polyalkyl-2-cyanoacrylates
Polyurethanes (PU)
Polyvinylalcohol (PVA)
Polypeptides
Poly-β-malic acid (PMLA)
Poly-β-alkanoic acids
Reference:
P. Tormala, S. Vainionpan and P. Rokkanen in IVA's Beijer Symposium “Biomaterials and Biocompatibility”. Stockholm, Sweden, Aug. 25-26, 1987.

In addition to the exemplary polymers and copolymers set forth in Table 1, other bioabsorbable materials may be suitable for use in various embodiments of surgical fasteners of the present invention. Such other bioabsorbable materials include, but are not limited to bioceramics and bioceramic/polymer composite materials.

Exemplary ceramic materials (bioceramics), which are tissue compatible and/or which form chemical bonds with bone tissue and/or which promote the growth of bone tissue and thus may be applicable to the present invention, are e.g. calcium phosphate: apatites like hydroxyapatite, HA, Ca₁₀(PO₄)₆(OH)₂; fluoroapatites, tricalcium phosphates (TCP) and dicalcium phosphates (DCP); magnesium calcium phosphates, S-TCMP; mixtures of HA and TCP; aluminium oxide ceramics; bioglasses like SiO₂—CaO—Na₂O—P₂O₅, e.g. Bioglass 45S (structure: SiO₂ 45 wt-%, CaO 24.5%, Na₂O 24.5% and P₂O₅ 6%); and glass ceramics with apatites, e.g. MgO 4.6 wt-%, CaO 44.9%, SiO₂ 34.2%, P₂O₅ 16.3% and CaF 0.5% and calcium carbonate.

One preferred material for bioabsorbable threaded component 105 of the present invention is poly (L-Lactide), and the preferred chemical spectfications for raw poly-lactide acid employed forbioabsorbable threaded components 105 are set forth herein in Table II.

TABLE II
Raw Poly-Lactic Acid Specifications

| | |
|---|---|
| Residual Tin (Stannous octoate): | Less than 200 ppm |
| Residual Metals (FE, Cr, Ni, Pb): | Less than 50 ppm each |
| Residual Lactide Dimer: | Less than 1% |
| Intrinsic Viscosity: | 6.5-8.5 dL/g in chloroform at 25° C. |

In certain embodiments of the present invention, a bioabsorbable threaded component 105 may preferably be formed by an injection molding process, and the preferred characteristics of the bioabsorbable threaded component thusly formed are set forth below in Table III.

TABLE III
Bioabsorbable Threaded Component Preferred Physical
Characteristics

| | |
|---|---|
| Ultimate Tensile Strength: | 9,000-15,000 psi |
| Tensile Modulus: | 330,000-530,000 psi |
| Maximum Bending Strength: | 12,900-20,900 psi |
| Bending Modulus: | 417,000-617,000 psi |
| Intrinsic Viscosity: | 2.0-4.5 dL/g in chloroform at 25° C. |

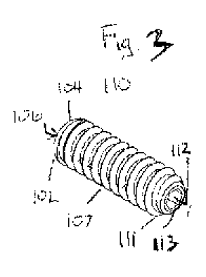

FIG. 3 provides a detailed view of an embodiment of a proximal metallic threaded component 110 of the biometal surgical fixation screw 100 of FIG. 1, in which the proximal metallic threaded component 110 comprises a distal end 111, a proximal end 102, and outer threads 107. In the embodiment shown in FIG. 3, the distal end 111 of the proximal metallic threaded component 110 comprises a keyed opening 113 continuous with a female keyed recess 112 axially located and extending within at least a portion of the length of the proximal metallic threaded component 110. The female keyed recess 112 is sized and keyed to receive the axial male keyed projection 101 of the bioabsorbable threaded component 105 of FIG. 2, such that rotational torque applied to the proximal metallic threaded component 110 will be uniformly transmitted and will cause equal rotation of an attached bioabsorbable threaded component 105.

Also in the embodiment shown in FIG. 3, the proximal end 102 of the proximal metallic threaded component 110 comprises a keyed opening 104 continuous with a female keyed recess 106 axially located and extending within at least a portion of the length of the proximal metallic threaded component 110, similar in hexagonal shape as the distal keyed female recess 112. The female keyed recess 106 and keyed opening 104 are sized and keyed to receive the keyed detachable rotational deployment tool 115 (shown in FIG. 1). A continuous uniform inner diameter that spans the entire length of the screw is shown in Section A-A of FIG. 1.

As disclosed herein in FIGS. 2 and 3, the axial male keyed projection 101 of the bioabsorbable threaded component 105 and the female keyed recess 106, keyed opening 104, keyed opening 113 and female keyed recess 112 may be keyed and sized in any operable form. In some preferred embodiments of the present invention, such keying may be based on a hex drive configuration.

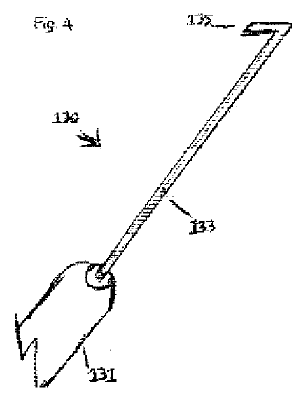

FIG. 4 shows an embodiment of a calibrated depth gauge 130 according to the present invention, comprising a handle 131, a calibrated shaft 133, and an angulated distal tip 135. In various embodiments of the present invention, the length of the calibrated shaft 133 may vary from about 10 mm to about 200 mm, with marked calibrations thereupon at intervals varying from 1 mm to 10 mm. In use, an operator uses the handle 131 and inserts the shaft 133 until the angulated distal tip 135 rests against the deepest extent of a cavity to be measured, allowing the operator to read the measurement on the calibrated shaft 133.

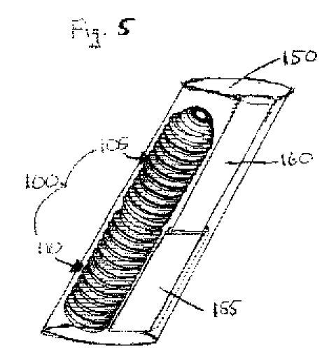

FIG. 5 is a perspective view of an exemplary use of a biometal surgical fixation screw according to the present invention securing a bone-tendon graft in a tibial tunnel in a procedure to reconstruct the anterior cruciate ligament (ACL) in a knee.

In the example shown in FIG. 5, a biometal fixation screw 100 is used to secure the tibial side of a patellar tendon bone (PTB) graft 160 during ACL reconstruction. The biometal fixation screw 100 comprises a distal bioabsorbable threaded component 105 joined with a proximal metallic threaded component 110 that are deployed together via a reverse taper fit using a tapered screw driver [not shown in FIG. 5]. The bioabsorbable threaded component 105 and metallic threaded component 110 each are provided in several length and diameter sizes to accommodate variations in bone block and tendon lengths and tunnel widths. This provides precise fixation of both the bony graft 155 and soft tissue tendon graft 160 portions of the patellar tendon bone graft within the tibial runnel 150. Also the bioabsorbable threaded component 105 is designed to be advanced to the tibial plateau allowing true joint line fixation of the graft and decreased extravasation of synovial fluid within the tibial tunnel 150 (which can prevent desired tendon to bone healing).

After determining tibial tunnel 150 length and creation of the tibial tunnel 150 using standard techniques, a custom depth gauge (see FIG. 4) is used to measure the anterior portion of the tibial tunnel length. The length of the tibial bone block 155 is known. After passing the PTB graft 160 and securing the femoral side with any number of techniques; the amount of bone and tendon lying within the tibial tunnel 150 are determined using the previous measurements. For example, if tibial tunnel length is 45 mm and bone block length is 25 mm, the amount of tendon within the tibial tunnel is 20 mm. Therefore the length of the metallic threaded component 110 is 25 mm which is mated with a 20 mm long bioabsorbable threaded component 105 and used to secure the graft. The metallic threaded component 110 will always follow the bioabsorbable threaded component 105 into the tibial tunnel using a standard antegrade technique over a guide wire with the bioabsorbable threaded component 105 and metallic threaded component 110 pressfit and driven together as one unit with the screwdriver. Screw diameter will ultimately be determined by physician preference; however the metallic threaded component 110 should be no smaller than 1 mm less than the tunnel 150 diameter. The bioabsorbable threaded component 105 may be the same size in diameter as the metallic threaded component 110 but may also be equal in diameter to the tibial tunnel 150. or smaller The fixation screw 100 is advanced while holding tension on the graft and performing a posterior drawer on the tibia.

Placement of the guide wire anterior to the graft can be confirmed arthroscopically prior to deploying the screw. During screw advancement, arthroscopic confirmation prevents deployment of the bio screw past the tibial plateau and into the joint.

Although the foregoing embodiments of the present invention have been described in some detail by way of illustration and example for purposes of clarity and understanding, it will be apparent to those skilled in the art that certain changes and modifications may be practiced within the spirit and scope of the present invention. Therefore, the description and examples presented herein should not be construed to limit the scope of the present invention, the essential features of which are set forth in the appended claims.

We claim:

1. Surgical fastener devices substantially as described in the specification and drawings herein.

2. Methods of fabricating surgical fastener devices substantially as described in the specification and drawings herein.

3. Methods of using surgical fastener devices substantially as described in the specification and drawings herein.
